(19) United States
(12) Patent Application Publication
(10) Pub. No.: US 2009/0054412 A1
(43) Pub. Date: Feb. 26, 2009

(54) Treatment of Sleep Disorders

(76) Inventors: John Alan Kemp, Basel (CH); Ian Michael Hunneyball, Abingdon (GB); Timothy Tasker, Isleworth (GB)

Correspondence Address: FITZPATRICK CELLA HARPER & SCINTO, 30 ROCKEFELLER PLAZA, NEW YORK, NY, 10112, US

(21) Appl. No.: 11/841,262

(22) Filed: Aug. 20, 2007

(51) Int. Cl. A61K 31/5517; A61P 25/00

(52) U.S. Cl. 514/220

(57) ABSTRACT

A method for treating various types of insomnia is provided using 7-chloro-3-(5-dimethylaminomethyl-[1,2,4]oxadiazol-3-yl)-5methyl-4,5-dihydro-imidazol[1,5,-a][1,4]benzodiazepine-6-one or its pharmaceutically acceptable salt.

### BACKGROUND OF THE INVENTION

### Field of the Invention

Insomnia is one of the most common complaints in general medical practice. Approximately 10% to 15% of adults suffer from chronic insomnia, and an additional 25% to 35% have transient or occasional insomnia. Transient insomnia is an insomnia that is present for one to several days, and is less than one week in duration. Short term insomnia is an insomnia of one to three weeks in duration. Chronic insomnia is typically accepted to involve episodes greater than three (3) weeks in duration (Roth, Int. J. Clin. Pract. 2001; (Suppl.):3-8).

Generally, as discussed in detail by Russell P. Rosenberg in “Sleep Maintenance Insomnia: Strengths and Weaknesses of Current Pharmacologic Therapies,” Annals of Clinical Psychiatry, 18[1]:49-56, 2006, which is incorporated herein by reference, patients with insomnia are further divided into groups based on when their sleep difficulty most often occurs. The three recognized categories of insomnia are (1) sleep onset insomnia (difficulty in falling asleep); (2) sleep maintenance insomnia (difficulty staying asleep); and (3) terminal insomnia (early-morning awakenings coupled with an inability to return to sleep). Terminal insomnia is sometimes referred to as sleep offset insomnia. These symptoms may occur singly or in combination, as is the case in many patients with chronic insomnia, which may result from several different etiologies. Patients often have several sleep complaints simultaneously and experience a gamut of sleep disturbances, including prolonged latency to sleep onset, increased time awake during the sleep period and reduced total sleep time.

There are various medications that have been used to treat insomnia. The early type of insomnia drugs are what have come to be known as classic benzodiazepines. These benzodiazepines exert their pharmacological actions by interacting with the benzodiazepine binding sites associated with the GABA_{A} receptor. GABA_{A} receptors are ligand-gated ion channels, and functional receptors are made up from combinations of different subunit proteins. Subunits are divided in three main classes of alpha (α), beta (β) and gamma (γ) subunits. GABA_{A} receptors that have a benzodiazepine binding site are formed from either α₁, α₂, α₃ or α₅ subunits in combination with β and γ₂ subunits (Paul J. Whiting, DDT Vol. 8, No. 10, May 2003).

The important allosteric modulatory effects of drugs acting at the benzodiazepine site were recognized early and the distribution of activities at different receptor subtypes has been an area of intense pharmacological discovery. Agonists that act at the benzodiazepine site are known to exhibit anxiolytic, sedative, and hypnotic effects. However, while some classic benzodiazepines, which are considered full agonists at the GABA_{A} receptor benzodiazepine site, are generally regarded as being effective at inducing and maintaining sleep, which is believed to be due to their relatively long half-lives ranging from 10-40 hours, they were found to produce undesirable residual effects. These may include cognitive impairment, excessive sedation, ataxia, potentiation of ethanol effects and a tendency for tolerance and drug dependence. A particular problem with classic benzodiazepines is rebound insomnia, manifested by restlessness and somnipathy, which emerges on withdrawal. Furthermore, the quality of sleep that is induced by these compounds is unphysiological. Classic benzodiazepines typically reduce slow wave sleep (SWS), REM sleep and generally adversely affect sleep architecture. One of the reasons for these undesirable side effects was deemed to be associated with the afore-mentioned relatively long half-life of the classic benzodiazepines.

In order to overcome these problems, agents with shorter half-lives have been investigated. Examples of such agents include the so-called non-benzodiazepines, such as zolpidem and zalepon, that also act as full agonists at the GABA_{A} receptor benzodiazepine site. However, while these newer agents are generally effective in reducing time to sleep onset (i.e., decreasing sleep latency), they have been found to be less effective at improving sleep maintenance, as well as treating terminal insomnia.

Sleep maintenance difficulties can be quantified using Polysomnography (PSG). When quantifying sleep maintenance difficulties via PSG, wake time after sleep onset (WASO) and number of awakenings (NAW) are the most commonly utilized parameters. WASO is a robust measure of sleep maintenance, as it represents the total amount of time spent awake after the person has reached persistent sleep, while NAW represents only the number of awakenings the person has experienced. Therefore, a person may wake only once during the night (NAW), but may spend 3 hours awake (WASO), so the latter measure more closely reflects the level of disturbance.

Difficulty with maintaining sleep is common in patients with medical and psychiatric disorders, as well as in patients with primary insomnia, and it occurs with more frequency than sleep onset problems in certain population groups. However, it is widely recognized that currently used medications fall short when it comes to safely and effectively addressing sleep maintenance problems.

### SUMMARY OF THE INVENTION

The present invention provides an effective method for treating sleep maintenance insomnia and/or terminal insomnia, each of which can be associated with transient, short term, chronic, primary and secondary insomnia. Specifically, the present invention provides a method for decreasing wake time after sleep onset (WASO), increasing total sleep time (TST), reduced total wake time per hour and/or reducing early-morning awakenings. All of these advantages can be achieved while reducing latency to sleep onset and/or latency to persistent sleep, thus also effectively treating sleep onset insomnia. Accordingly, the present invention provides an effective method for treating various types of insomnia.

This method involves the use of 7-chloro-3-(5-dimethylaminomethyl-[1,2,4]oxadiazol-3-yl)-5-methyl-4,5-dihydro-imidazo[1,5-a][1,4]benzodiazepine-6-one, which is represented by formula (II) below, or a pharmaceutically acceptable salt thereof to treat sleep maintenance insomnia and/or terminal insomnia, as well as the above-outlined specific conditions:

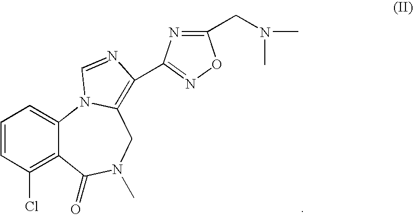

Preferably, the amount of the compound of formula (II) or its pharmaceutically acceptable salt that is administered for the treatment is from about 0.5 mg to about 5 mg. The treatment amount may be from about 1.0 mg to about 4.5 mg, from about 1.5 mg to about 4 mg, from about 2 mg to about 3.5 mg, from about 2.5 mg to about 3 mg, or any range among all of the above-listed amounts. For example, the treatment amount is from about 0.5 mg or about 1.5 mg to about 5 mg, about 4.5 mg, about 4 mg, about 3.5 mg, about 3 mg or about 2.5 mg. More preferably, the amount is from about 1 mg to about 3 mg, yet more preferably from about 1.5 mg to about 2.5 mg.

Accordingly, a particularly preferably pharmaceutical composition for the treatment in accordance with the present invention contains from about 0.5 mg to about 5 mg of the compound of formula (II). More preferably, the pharmaceutical composition will be in a unit dosage form comprising 0.5 mg, 1.5 mg, 2 mg, 2.5 mg, 3 mg, 3.5 mg, 4 mg, 4.5 mg or 5 mg of the compound of formula (II).

The subjects to be treated in accordance with the present invention are humans.

As used herein, latency to persistent sleep (LPS) is defined as the time from “lights out” to the beginning of 10 uninterrupted minutes of sleep.

Persistent sleep is defined as 10 uninterrupted minutes of sleep after initial sleep onset.

Wake after sleep onset (WASO) is defined as the total amount of time spent awake after the onset of persistent sleep over a fixed 8-hour period in bed (captures total duration of lost sleep after at least 1 awakening).

Number of awakenings (NAW) is defined as the return to an awake state (number of wake periods lasting at least 1 minute occurring after the onset of persistent sleep).

Total sleep time (TST) is defined as the total time asleep over a fixed 8-hour period.

Sleep efficiency index is a ratio of TST to total time in bed, i.e., a percentage of time spent asleep. Total time in bed is typically 8 hours for study purposes.

Sleep architecture refers to the changes in the stages of sleep during the sleep period. Typically, in healthy humans, sleep stages occur in cycles lasting about 90 to about 120 minutes each. Four to five such cycles occur during a typical night of sleep. During the first half of the night, the healthy individual typically passes from wakefulness briefly into stage I sleep and then to stages II, III, and IV. Stages II and III reappear, after which rapid eye movement (REM) sleep is observed for the first time. During the second half of the night, stage II and REM sleep alternate.

Slow wave sleep (SWS) is stage III and IV sleep. It is characterized by a transition to an electroencephalogram (EEG) with high amplitude delta EEG waves (1.5 to 3 Hz).

As used herein, AUC is the area under the drug plasma concentration versus time curve from time zero to infinity. Cₘₐₓ is the maximum observed plasma concentration of the drug from time zero to infinity.

### BRIEF DESCRIPTION OF THE DRAWINGS

FIG. 1 is a plot showing concentration-dependent stimulation of currents elicited by GABA (EC₃₋₅) by the compound of formula (II) at α₁β₂γ₂, α₂β₂γ₂, α₃β₂γ₂ and α₅β₂γ₂ GABA_{A} receptors expressed in Xenopus oocytes. Data is shown as mean±SEM.

FIG. 2 is a plot showing concentration-dependent stimulation of currents elicited by GABA (EC₃₋₅) by the compound of formula (II) at α₁β₂γ₂, α₂β₂γ₂, α₃β₂γ₂ and α₅β₂γ₂ GABA_{A} receptors expressed in Xenopus oocytes. Stimulation is standardized to the one observed using 1 μM diazepam in the same batch of oocytes. Data is shown as mean±SEM.

FIG. 3 is a plot showing concentration-dependent stimulation of currents elicited by GABA (EC₃₋₅) by zolpidem at α₁β₂γ₂, α₂β₂γ₂, α₂β₃γ₂ and α₅β₂γ₂ GABA_{A} receptors expressed in Xenopus oocytes. Data is shown as mean +SEM.

FIG. 4 is a plot showing concentration-dependent stimulation of currents elicited by GABA (EC₃₋₅) by zolpidem at α₁β₂γ₂, α₂β₂γ₂, α₂β₃γ₂ and α₅β₂γ₂ GABA_{A} receptors expressed in Xenopus oocytes. Stimulation is standardized to the one observed using 1 μM diazepam in the same batch of oocytes. Data is shown as mean±SEM.

FIG. 5 shows the study design used in Example 3.

FIG. 6 is a chart showing latency to persistent sleep in Example 3.

FIG. 7 is a chart showing total sleep time in Example 3.

FIG. 8 is a chart showing wake after sleep onset in Example 3.

FIG. 9 is a chart showing wake after sleep onset in the first and second halves of the night in Example 3.

FIG. 10 is a chart showing the percent reduction (vs. placebo) in wake after sleep onset in the first and second halves of the night in Example 3.

FIG. 11 is a chart showing total wake time for each hour of the night in Example 3.

FIGS. 12 and 13 are charts showing patient reported sleep quality in Example 3.

FIG. 14 is a chart showing sleep architecture in accordance with Example 3.

FIG. 15 is a chart showing patient reported residual sedation effects in accordance with Example 3.

### DETAILED DESCRIPTION OF THE INVENTION

One of the major challenges in treating insomnia is to develop a drug that induces sleep quickly, helps individuals remain asleep and allows them to awaken feeling refreshed rather than hung over. The present invention meets this challenge and provides effective methods for treating all types of insomnia. In particular, the present invention provides a method for treating maintenance and/or terminal insomnia by administering to a human in need of this treatment an effective amount of the compound of formula (II) or a pharmaceutically acceptable salt thereof:

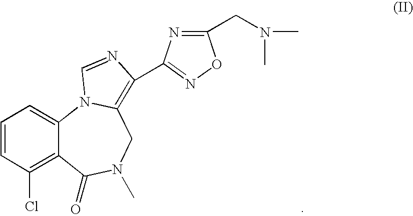

The compound of formula (II) can be prepared in accordance with the methods described in U.S. Pat. No. 6,391,873, which is incorporated herein by reference. It has been disclosed as useful for treating acute and chronic anxiety disorders.

As disclosed in U.S. Pat. No. 5,665,718, this type of compound is deemed to display sedative activity that sets in very rapidly, but lasts only a relatively short period of time. Accordingly, the compound of formula (II) would not be expected to be beneficial in the treatments of maintenance and terminal insomnia.

Nonetheless, the compound of formula (II) was surprisingly found to be effective for treatment of not only sleep onset insomnia in humans, but also maintenance insomnia and terminal insomnia, even when the administration amount was significantly low, on the order of about 0.5 mg to about 5 mg, particularly from about 1.5 mg to about 2.5 mg. The surprising nature of these results is further supported by the finding that the compound of formula (II) has a relatively short half-life of about 3-4 hours, akin to the conventional insomnia treatment agents having relatively short half-lives, which were found lacking effectiveness in sleep maintenance. The efficacy of the compound of formula (II) for the treatment of various types of insomnia was not found to be improved when the administration amount exceeded about 5 mg, and residual sedation effects were noted at higher doses. Conventional insomnia agents, such as zolpidem, trazodone and zalepon, were found to be less than effective for treating maintenance and terminal insomnia even when administered in amounts that are at least twice that of the compound of formula (II) (see Roth, Int. J. Clin. Pract. 2001; (Suppl.):3-8).

Pharmaceutically acceptable salts for the compound of formula (II) can be prepared by standard techniques that will be familiar to the person skilled in the art. Suitable pharmaceutically acceptable salts include those with inorganic or organic acids. Examples of such salts are the hydrochlorides, hydrobromides, sulfates, nitrates, citrates, acetates, maleates, succinates, methanesulphonates, p-toluenesulphonates and the like.

The compound of formula (II) achieves its sedative effects by positive allosteric modulation of GABA_{A} receptors via the benzodiazepine site. However, unlike conventional agents for treating insomnia that act at the benzodiazepine site, the compound of formula (II) is only a partial agonist, i.e., it produces a lower maximum potentiation of the GABA_{A} receptor. Thus, unexpectedly, it was found that even a partial agonist can be used for the treatment of maintenance and terminal insomnia.

The ability to allosterically stimulate currents elicited by GABA (EC₃₋₅) was determined for the compound of formula (II), zolpidem and diazepam at the rat GABA_{A} receptors of the subunit composition α₁β₂γ₂, α₂β₂γ₂, α₃β₂γ₂ and α₅β₂γ₂. 7-Chloro-3-(5-dimethylaminomethyl-[1,2,4]oxadiazol-3-yl)-5methyl-4,5-dihydro-imidazol[1,5,-a][1,4]benzodiazepine-6-one was used to test the effects of the compound of formula (II):

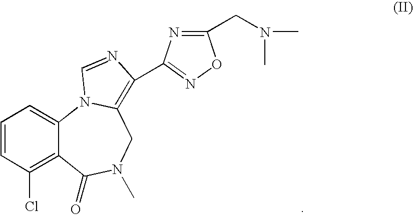

Similar procedures were chosen as for the investigation of other GABA_{A} receptor subunit specific substances (e.g., Baur et al., 2005; Mol. Pharmacol. 68, 787-792).

Expression in Xenopus oocytes

Lobes of the ovary containing the follicles were obtained by surgical procedures from female Xenopus laevis. Follicles were singled out using a platinum loop. Fifty nL of cRNA solution at a ratio of αₓ:β₂:γ₂ of 1:1:5 (3.3-10 nM αₓ (x=1,2,3,5); 3.3-10 nM β₂; 16.7-50 nM γ₂) (Boileau et al., 2002; Neuropharmacology 43, 695-700) were microinjected into Xenopus follicles. Several hours after microinjection the follicles were freed of follicular layers and adhering connective tissue by a collagenase/hypertonic shock procedure (Sigel, 1987; J. Physiol.(Land) 386, 73-90). Oocytes were kept at constant 18° C. until measurement (1-4 days) in a modified Barth solution (88 mM NaCl, 1 mM KCl, 2.4 mM NaHCO₃, 10 mM Hepes-NaOH (pH 7.5), 0.82 mM MgSO₄×7H₂O, 0.34 mM Ca(NO₃)₂×4H₂O, 0.41 mM CaCl₂×2H₂O, 100 U Penicillin/mL, 100 μg Streptomycin/mL, sterile filtered).

### Electrophysiological Investigation

Currents were measured using a home-built amplifier in combination with a xy-recorder or were digitized using a MacLab/200 (AD Instruments) and stored on a computer. Xenopus oocytes were voltage clamped using the two-electrode voltage clamp technique (electrode resistance about 0.8 MΩ) at −80 mV. The medium contained 90 mM NaCl, 1 mM KCl, 1 mM MgCl₂, 1 mM CaCl₂, 10 mM Na-Hepes (pH 7.4) and 0.5% DMSO.

GABA was applied for 20-50 seconds without or in combination with other drugs and a washout period of 4 minutes was allowed to ensure full recovery from desensitization, which was experimentally determined. The perfusion solution (6 mL/min) was applied through a glass capillary with an inner diameter of 1.35 mm, the mouth of which was placed about 0.4 mm from the surface of the oocyte. The rate of solution change under our conditions has been estimated 70% within less than 0.5 s (Sigel et al., 1990; Neuron 5, 703-711). The entire perfusion system and the assay chamber were cleaned between drug applications by washing with DMSO.

### Data Handling

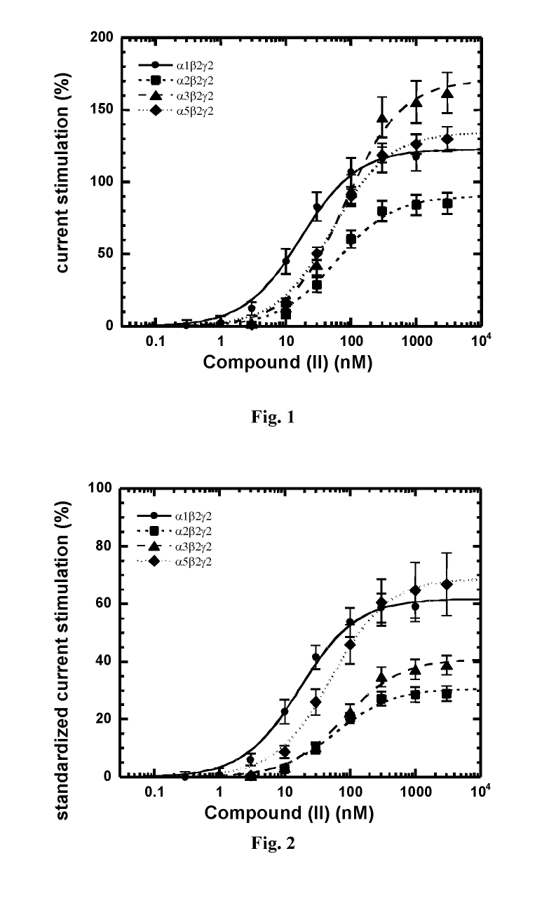

Data is given as mean±SD, except in the figures where data is shown for clarity as mean±SEM. Current stimulation was calculated as follows: stimulation (%)=((I_{(GABA+modulator)}−I_{(GABA)})/I_{(GABA)})×100%, where I is the current amplitude. Where indicated, the stimulation was standardized to the stimulation by 1 μM diazepam (100%). To obtain FIGS. 1-4, values for current stimulation obtained at a given concentration of modulator at a given subunit combination were averaged. The data points were fitted with the equation stimulation=efficacy/(1+(potency/concentration of modulator)). The values given in the Tables (Summary) are obtained by fitting individual curves and subsequently averaging efficacy and potency.

### RESULTS

### Compound of Formula (II)

GABA (EC₃₋₅) was applied to an oocyte expressing α₁β₂γ₂ GABA_{A} receptors several times until the current response was stable. The GABA (EC₃₋₅) refers to the effective concentration of GABA, which produces a response that is 3-5% of the maximal response to high concentrations of GABA. Such a low concentration of GABA is chosen in order to better see the potentiating effect of positive allosteric modulators.

GABA was then applied in combination with various concentrations of the compound of formula (II) between 0.3 nM and 3,000 nM to produce a cumulative concentration response curve. This resulted in a concentration-dependent potentiation of the GABA response as plotted in FIGS. 1 and 2. In each batch of oocytes the stimulation by 1 μM diazepam was determined in five ocytes, extent of stimulation averaged and defined as 100%. Where indicated, stimulation by the compound of formula (II) in each batch of ocytes was expressed as a percentage of this value in the corresponding batch.

Concentration response curves were also performed with oocytes expressing α₂β₂γ₂, α₃β₂γ₂, or α₅β₂γ₂, after establishing the optimal concentration range as indicated above. The compound of formula (II) performed as a partial positive allosteric modulator. At concentrations <100 nM, the compound of formula (II) showed preference for α₁β₂γ₂ GABA_{A} receptors in comparison to α₅β₂γ₂, α₃β₂γ₂ and α₂β₂γ₂. FIG. 1 shows the dose dependent stimulation of currents elicited by GABA at α₁β₂γ₂, α₂β₂γ₂, α₃β₂γ₂, and α₅β₂γ₂ before and FIG. 2 after standardization to the stimulation by 1 μM diazepam (100%). Averaged data of the individual curves summarizing the effects of the compound of formula (II) are shown below for unstandardized and standardized stimulation.

TABLE 1
Summary for Compound of Formula (II)

| | | | Standardized |
|---|---|---|---|
| Receptor | Potency | Efficacy | Efficacy |
| α₁β₂γ₂ | 18 ± 6 nM (n = 4) | 123 ± 19% (n = 4) | 62 ± 10% (n = 4) |
| α₂β₂γ₂ | 62 ± 21 nM (n = 5) | 90 ± 16% (n = 5) | 31 ± 6% (n = 5) |
| α₃β₂γ₂ | 84 ± 15 nM (n = 5) | 171 ± 33% (n = 5) | 41 ± 8% (n = 5) |
| α₅β₂γ₂ | 53 ± 6 nM (n = 5) | 135 ± 16% (n = 5) | 69 ± 23% (n = 5) |

### Zolpidem

GABA (EC₃₋₅) was applied to an oocyte expressing α₁β₂γ₂ GABA_{A} receptors several times until the current response was stable. Subsequently, GABA was applied in combination with various concentrations of zolpidem between 1 and 10,000 nM. Concentration response curves were performed twice with the same batch of oocytes and twice with an independent batch of oocytes.

In each batch of oocytes the stimulation by 1 μM diazepam was determined in five oocytes, extent of stimulation averaged and defined as 100%. Where indicated, stimulation by zolpidem in each batch of oocytes was expressed as percentage of this value in the corresponding batch.

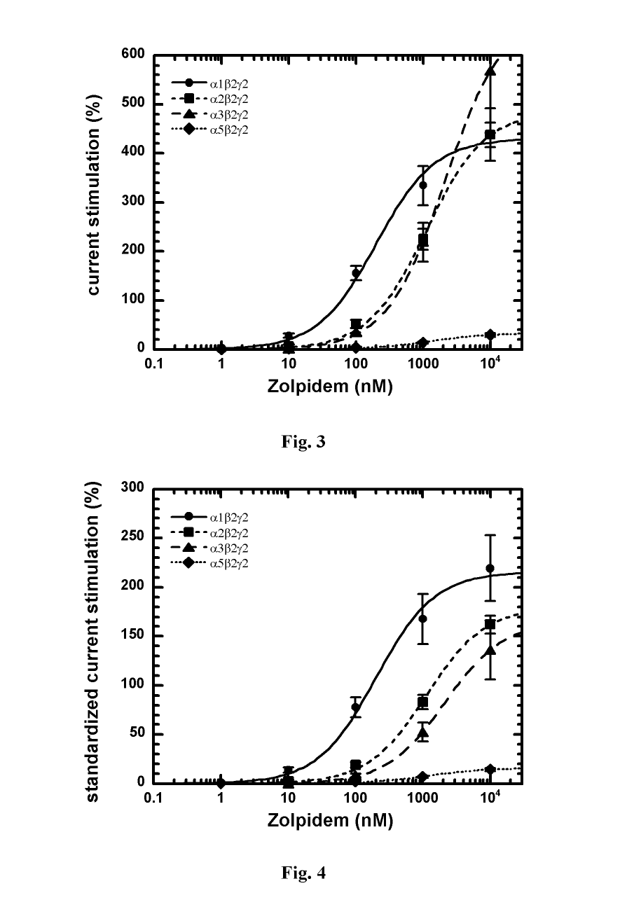

Concentration response curves were also performed with oocytes expressing α₂β₂γ₂, α₃β₂γ₂, or α₅β₂γ₂. As expected, zolpidem showed a higher affinity at α₁β₂γ₂ GABA_{A} receptors in comparison to α₂β₂γ₂, α₃β₂γ₂, and α₅β₂γ₂. FIG. 3 shows the dose dependent stimulation of currents elicited by GABA at α₁β₂γ₂, α₂β₂γ₂, α₃β₂γ₂, and α₅β₂γ₂ before and FIG. 4 after standardization to the stimulation by 1 μM diazepam (100%). Qualitatively similar data have been published before (Sanna et al. 2002; Eur. J. Pharmacol. 451, 103-110). Averaged data of the individual curves summarizing the effects by zolpidem are shown below for unstandardized and standardized stimulation.

TABLE 2
Summary for Zolpidem (n = 5 each)

| Receptor | Potency | Efficacy | Standardized Efficacy |
|---|---|---|---|
| α₁β₂γ₂ | 191 ± 34 nM | 429 ± 120% | 215 ± 74% |
| α₂β₂γ₂ | 1135 ± 312 nM | 484 ± 60% | 179 ± 23% |
| α₃β₂γ₂ | 2021 ± 495 nM | 691 ± 365% | 166 ± 88% |
| α₅β₂γ₂ | 1260 ± 744 nM | 33 ± 12% | 16 ± 4% |

### Diazepam

Current stimulation by diazepam was determined in each batch of oocytes as follows. GABA (EC₃₋₅) was applied until a stable response was obtained. Subsequently, GABA was applied in combination with 1 μM diazepam. Stimulation at the same subunit combination in different batches of oocytes was not statistically different, in each case. Stimulation by 1 μM diazepam at α₁β₂γ₂ amounted to 223+28% (n=5) and 178+20% (n=5) in two different batches of oocytes. Stimulation at α₂β₂γ₂ amounted to 264+61% (n=5), 280+71% (n=5) and 318+62% (n=5) in three different batches of oocytes. Stimulation at α₃β₂γ₂ amounted to 417+85% (n=5) and 417+144% (n=5) in two different batches of oocytes. Stimulation at α₅β₂γ₂ amounted to 237+97% (n=5) and 160+4% (n=5) in two different batches of oocytes.

The results obtained for zolpidem are comparable to those achieved by Sanna et al 2002 (referenced above) in previous experiments and show an efficacy of 215% relative to diazepam at α₁β₂γ₂ GABA_{A} receptors. Zolpidem therefore acts as a positive allosteric modulator with high intrinsic activity, i.e., acts a full agonist. The compound of formula (II) showed a lower intrinsic activity, i.e., acts as a partial agonist.

Low intrinsic activity of the compound of formula (II) means that potentiation of the response mediated by GABA_{A} receptors is limited even at high levels of receptor occupancy, which could be achieved with high concentrations of the compound of formula (II). PET studies indicate zolpidem (20 mg) produces receptor occupancy of about 20% in man (Abadie et al., European Journal of Pharmacology, 295 (1996), 35-44), i.e., clinical dose (10 mg) is on the steep inflection part of the dose-response curve. The compound of formula (II), nevertheless, still produces sufficient potentiation of the GABA_{A} receptor to be highly effective for both sleep onset and maintenance. Excessive potentiation at higher doses is limited.

Producing lower stimulation at the individual receptor level is believed to be advantageous. Since the maximal potentiation of the GABA_{A} response produced by such a low efficacy agonist is limited by its intrinsic efficacy, no further potentiation of the GABA_{A} response is achieved beyond a certain plasma concentration. In a clinical setting, such a limit on the maximum potentiation of the GABA_{A} receptor mediated response provides an advantageous ability to avoid excess potentiation with increasing plasma concentrations.

As a result of partial agonist activity at the GABA_{A} receptor benzodiazepine site, the compound of formula (II) can also provide a more restful and improved quality of sleep by practically having no effect on sleep architecture, producing no impairment of slow wave sleep (SWS). Classic benzodiazepines, which act as full agonists, typically reduce SWS and generally adversely affect sleep architecture. This ability to produce improved quality sleep over a sustained period, whilst minimizing side effects, leads to the advantageous use of the compound of formula (II) for the treatment of various types of insomnia. In particular, various types of insomnia may be treated advantageously by achieving a maximal potentiation of the response mediated by the al subunit containing GABA_{A} receptors from only about 40% to about 90% using the compound of formula (II) or its pharmaceutically acceptable salts.

The potentiation of the GABA_{A} mediated response over time following the administration (e.g., oral) of the compound of formula (II) may be determined using a model. In this model, measured or predicted free plasma concentration following the dosing of the compound of formula (II) (assuming 50% plasma protein binding) as the clinically relevant drug concentration and the in vitro concentration-response data for GABA_{A} receptor potentiation as discussed above can be used to predict the percent potentiation of the response mediated by GABA_{A} α₁β₂γ₂ (α₁-containing) receptors over time after the administration. Specifically, the percent potentiation of the GABA_{A} α₁β₂γ₂ receptor mediated response for the compound of formula (II) can be calculated as follows:

% potentiation=Efficacy (maximal % potentiation of GABA_{A} α₁β₂γ₂ receptor)/[1+(EC50/concentration of the compound of formula (II))].

The compound of formula (II) and pharmaceutically acceptable acid addition salts thereof can be used as medicaments, for example in the form of pharmaceutical preparations. The pharmaceutical preparations are typically administered orally, for example, in the form of tablets, coated tablets, dragees, hard and soft gelatine capsules, solutions, emulsions or suspensions. The administration can, however, also be effected rectally, for example, in the form of suppositories, or parenterally, for example, in the form of injection solutions.

The compound of formula (II) and pharmaceutically acceptable acid addition salts thereof can be processed with pharmaceutically inert, inorganic or organic carriers for the production of pharmaceutical preparations, and the like. Lactose, corn starch or derivatives thereof, talc, stearic acid or its salts and the like can be used, for example, as carriers for tablets, coated tablets, dragees and hard gelatine capsules. Suitable carriers for soft gelatine capsules are, for example, vegetable oils, waxes, fats, semi-solid and liquid polyols and the like; although carriers are not necessary in the case of soft gelatine capsules. Suitable carriers for the production of solutions and syrups are, for example, water, polyols, sucrose, invert sugar, glucose and the like. Adjuvants, such as alcohols, polyols, glycerol, vegetable oils and the like, can be used for aqueous injection solutions of water-soluble acid addition salts of the compound of formula (II), but as a rule are not necessary. Suitable carriers for suppositories are, for example, natural or hardened oils, waxes, fats, semi-liquid or liquid polyols and the like.

The pharmaceutical preparations can also contain preservatives, solubilizers, stabilizers, wetting agents, emulsifiers, sweeteners, colorants, flavorants, salts for varying the osmotic pressure, buffers, coating agents or antioxidants. They can also contain other therapeutically valuable substances.

The compound of formula (II) or pharmaceutically acceptable salt thereof is preferably administered in the amount from about 0.5 mg to about 5 mg. More preferably, the administration amount is from about 1 mg to about 3 mg, even more preferably from about 1.5 mg to about 2.5 mg. The drug is preferably administered once daily in an oral dosage form shortly before the patient wants to sleep.

The present invention is further described by the following Examples. These Examples are indended to illustrate some of the embodiments of the present invention and are not to be construed as limitations thereon.

### EXAMPLE 1

A placebo controlled, randomized, double-blind, cross-over study of the effects of the compound of formula (II) was conducted using a road noise model using 12 healthy volunteers. Specifically, the volunteers were subjected to road traffic noise to imitate the effects of insomnia, and the medication was orally administered 5 minutes before the 11 pm bed time in 1.0 mg, 1.5 mg, 2 mg and 2.5 mg doses in the form of a hard gelatine capsule containing the powdered compound of formula (II). Measurements were then taken at 8, 10 and 12 hours after dosing.

The results of the study are summarized in Tables 3-5.

TABLE 3
Objective Efficacy

| | | |
|---|---|---|
| Sleep | Wake After Sleep Onset | |
| Maintenance | (WASO): | |
| | WASO full night (hrs 0-8) | Significantly reduced |
| | | (1.5, 2.0 & 2.5 mg) |
| | WASO 2nd half night (hrs 5-8) | Significantly reduced |
| | | (1.5, 2.0 & 2.5 mg) |
| | Total Sleep Time | Significantly improved |
| | | (2.0 & 2.5 mg) |
| | Sleep Efficiency Index | Significantly improved |
| | | (2.0 & 2.5 mg) |
| Sleep | Slow Wave Sleep (% and | Significantly increased |
| Architecture | duration) | (all 4 doses) |
| | Stage IV Sleep (% and duration) | Significantly increased |
| | | (all 4 doses) |

TABLE 4
Subjective Efficacy

| | | |
|---|---|---|
| Leeds Sleep | Quality of sleep | Significantly improved at all |
| Evaluation | | 4 doses |
| Questionnaire | Getting to sleep | (No effect)* |
| | Ease of awakening | No effect |
| | Early morning behaviour | No effect |
| | (clumsiness & tiredness) | |

*The road traffic noise model is considered clinically nondiscriminant for sleep onset measures

TABLE 5
Objective Residual Effects

| | | |
|---|---|---|
| Attention and | Sustained Attention to Response | No effect |
| Accuracy | (SART) | |
| | Rapid Visual Information Processing | No effect |
| | Continuous Tracking Task - deviation | No effect at |
| | | 8 h |
| | Continuous Tracking Task - reaction | No effect at |
| | time | 10 or 12 h |
| Memory | Sternberg Memory Task (STM) | No effect at |
| | | 8 or 12 h |
| | Word Recall - immediate (WRi) | Impaired at |
| | | 8 h* |
| | Word Recall - delayed (WRd) | No effect |
| Information | Critical Flicker Fusion Test | No effect |
| Processing | | |
| Sensory | | |
| Discrimination | | |
| Motor Coordination | Choice Reaction Time - Motor | No effect |
| | Component | |

*Immediate word recall was not tested at 10 and 12 hours

When the data was corrected for multiple comparisons, there was no impairment of performance on any of the cognitive or psychomotor tests 8 to 12 hours after the dose was administered. Any residual effects that were observed the morning after dosing were inconsistent. Residual effects did not appear to be dose or time related.

TABLE 6
Subjective Residual Effects at 2.5 mg

| | | |
|---|---|---|
| Subjective assessment (LARS) | Sedation | No impairment |
| | Mood | No impairment |
| | Coordination | No impairment |

### EXAMPLE 2

A single and repeat dose pharmacokinetic safety and pharmacodynamic study of the effects of the compound of formula (II) was conducted using healthy volunteers. The compound of formula (II) was administered orally in 1 mg, 1.5 mg, 2 mg and 2.5 mg doses via a hard gelatine capsule containing the compound in powder form.

The pharmacokinetic analysis of the results showed that the half-life of the compound of formula (II) is about 3.5 hours. There were no significant differences in the pharmacokinetic profile on day 14 after repeat dosing compared to day 1. Food was found to have little or no effect on the extent of the absorption of the compound of formula (II).

The analysis of the pharmacokinetic data also shows that onset, maintenance and/or terminal insomnia may be treated by administering the compound of formula (II) to achieve an AUC from about 17.5 ng·h/mL to about 600 ng·h/mL, from about 25 ng·h/mL to about 500 ng·h/mL or from about 25 ng·h/mL to about 400 ng·h/mL. For example, the AUC may be from about 52.5 ng·h/mL to about 360 ng·h/mL, from about 75 ng·h/mL to about 300 ng·h/mL, from about 75 ng·h/mL to about 240 ng·h/mL, from about 75 ng·h/mL to about 200 ng·h/mL, from about 75 ng·h/mL to about 150 ng·h/mL, from about 105 ng·h/mL to about 120 ng·h/mL, or any range among all of the above-listed AUC values. Preferably, the AUC is from about 75 ng·h/mL to about 240 ng·h/mL.

The treatment is also conducted to achieve a Cₘₐₓ from about 2.5 ng/mL to about 125 ng/mL, from about 7.5 ng/mL to about 75 ng/mL, from about 7.5 ng/mL to about 62.5 ng/mL, from about 7.5 ng/mL to about 37.5 ng/mL, from about 10 ng/mL to about 50 ng/mL, from about 12.5 ng/mL to about 45 ng/mL, from about 15 ng/mL to about 40 ng/mL, or any range among all of the above-listed Cₘₐₓ values. Preferably, the Cₘₐₓ is from about 15 ng/mL to about 45 ng/mL.

### EXAMPLE 3

A randomized, multicenter, double-blind, placebo-controlled crossover study was conducted to assess the efficacy of the 1.5 mg and 2.5 mg doses of the compound of formula (II) in the treatment of primary insomnia in adult patients. Specifically, one of the objectives of the study was to assess the efficacy of 1.5 mg and 2.5 mg doses on polysomnogram (PSG) and patient-reported measures of sleep. Also, the study was aimed at assessing the safety of 1.5 mg and 2.5 mg doses.

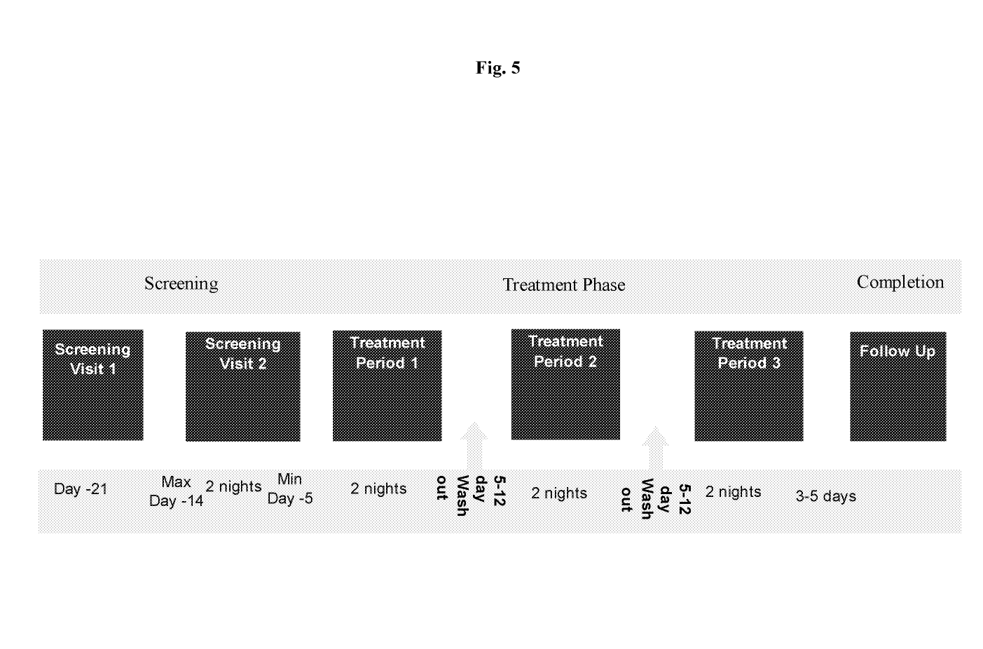

The study in this Example was performed for two consecutive nights with a 5-12 day washout between each period. The dosing was conducted 30 minutes before lights were turned out for the night via oral administration of a hard gelatine capsule containing the compound of formula (II) in powder form. PSG was taken for 8 hours from “lights out” on nights 1 and 2 of each treatment period. Centralized scoring of PSG was used. Testing for residual effects using the Digit Symbol Substitution Test (DSST) was performed at least 30 minutes after wake time (9 hours post dose). The overall study design is shown in FIG. 5.

The study was conducted using 67 subjects younger than 65 years of age (21 males, 46 females; mean age 45.1 yrs, range 23-64 yrs) with a documented diagnosis of primary insomnia (DSM-IV criteria). These subjects' typical bed time was between 9 pm and 1 am with at least 7 hours in bed. These subjects reported sleep latency of at least 45 minutes and total sleep time (TST) of not more than 6.5 hours in a sleep diary for at least 3 of 7 nights.

On screening using PSG for 2 nights, the patients showed latency to persistent sleep (LPS) of more than 20 minutes, with no nights showing LPS of less than 15 minutes. Mean wake time after sleep onset (WASO) of the patients was at least 40 minutes and mean TST was 240-420 minutes.

The top-line efficacy results of the study in Example 3 are shown in Table 7.

TABLE 7
Top-Line Efficacy Results

| | 1.5 mg vs. | 2.5 mg vs. |
|---|---|---|
| Parameter | placebo | placebo |
| Adjusted mean TST (min) | p < 0.0001 | p < 0.0001 |
| Adjusted mean WASO (min) | p < 0.0001 | p < 0.0001 |
| Adjusted mean LPS (min) | p < 0.0001 | p < 0.0001 |
| Adjusted mean total wake time, 2^{nd} half | p = 0.0008 | p < 0.0001 |
| (min) | | |

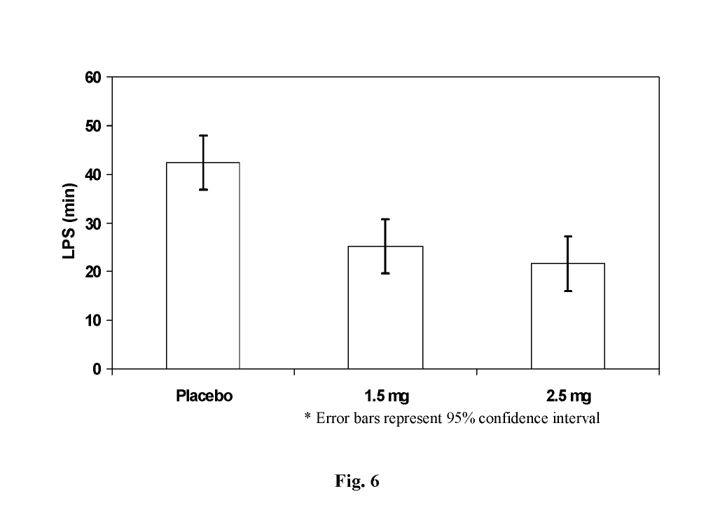

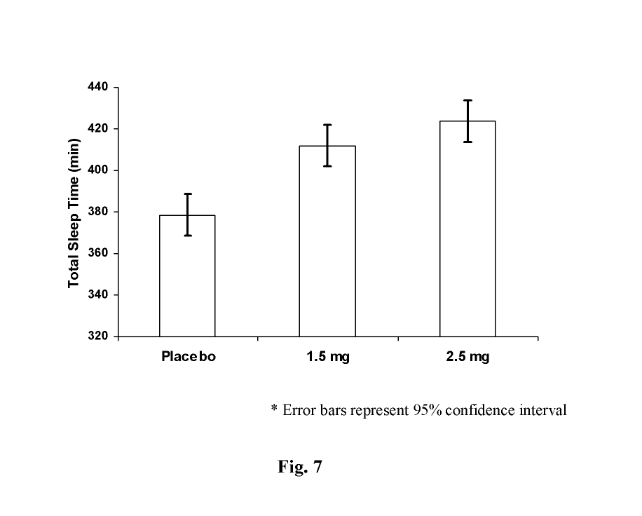

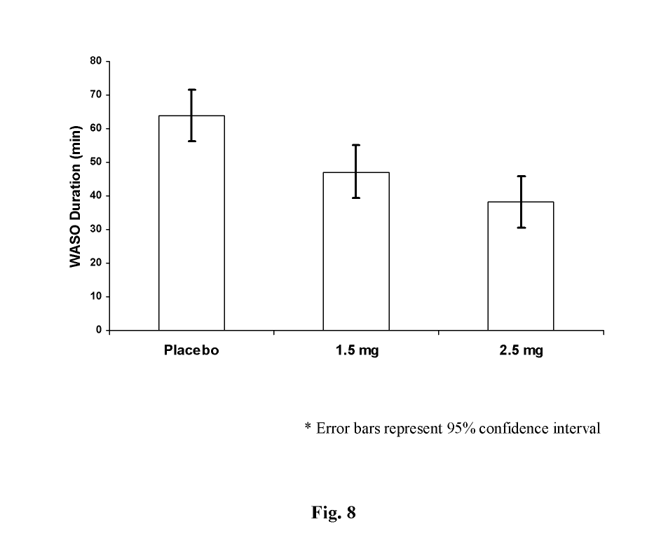

The compound of formula (II) showed robust effects on both sleep onset and sleep maintenance. Specifically, compared to a placebo, the 1.5 mg dose reduced LPS by 17.0 minutes (p<0.0001) and the 2.5 mg dose reduced LPS by 20.7 minutes (p<0.0001), as shown in FIG. 6. The 1.5 mg dose increased TST by 33.1 minutes (p<0.0001) and the 2.5 mg dose increased TST by 45.0 minutes (p<0.0001), as shown in FIG. 7, compared to a placebo. The 1.5 mg dose reduced WASO by 16.7 minutes (p<0.0001) and the 2.5 mg dose reduced WASO by 25.7 minutes (p<0.0001), as shown in FIG. 8, compared to a placebo.

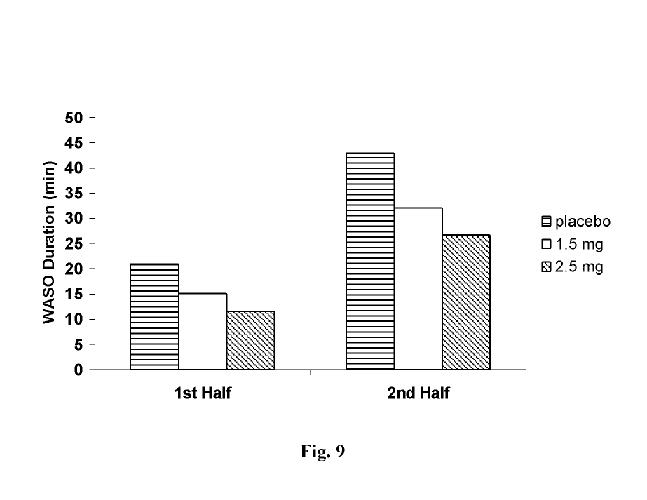

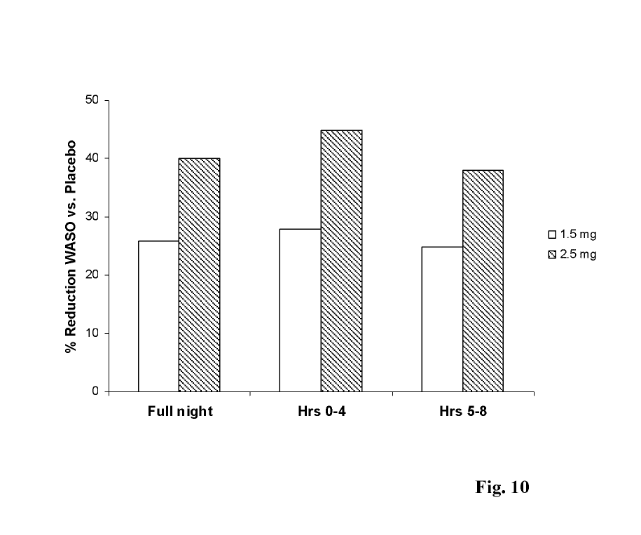

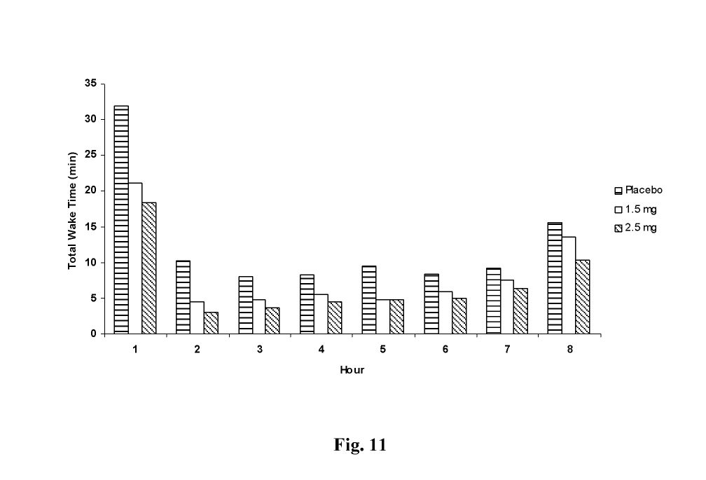

Importantly, as shown in FIGS. 9 and 10, these doses reduced WASO (p<0.0001) in the second half of the night (5-8 hours after “lights out”) compared to a placebo. Total wake time in hours 5-8 was significantly reduced, with the 1.5 mg dose showing a 10.8 minute reduction (p=0.0008) and the 2.5 mg dose showing a 16.2 minute reduction (p<0.0001). This demonstrates that the compound of formula (II) can be used to treat terminal insomnia and reduce early morning awakenings. In fact, a reduction in a total amount of time the subject was awake during each hour after dosing was observed, as shown in FIG. 11, with the 2.5 mg dose producing a statistically significant reduction each hour, except hour 7, where p was 0.0577 for the overall treatment effect (reduction in time awake during hour 7 almost reached statistical significance).

These results are particularly unexpected for an agent such as the compound of formula (II). Since it is only a partial agonist at the GABA_{A} receptor benzodiazepine site and its half-life is similar to some insomnia agents acting as full agonists at this site, which were found ineffective for the treatment of maintenance and terminal insomnia even when used in substantially larger amounts, the compound of formula (II) would be expected to be even less effective than these other agents. It has now been unexpectedly found that this is not the case.

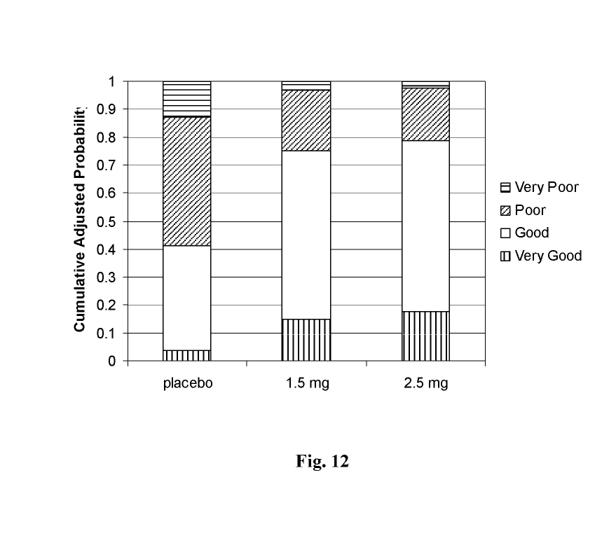

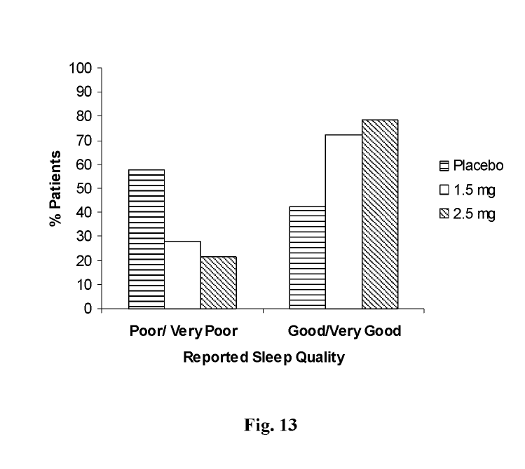

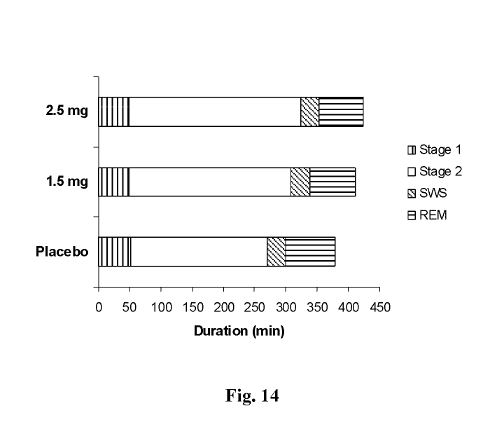

In addition to the improvements in the quantity of sleep, the subjects of the study also reported a marked improvement in sleep quality for both 1.5 and 2.5 mg doses, which is demonstrated in FIGS. 12 and 13. In particular, the compound of formula (II) was found to produce sleep architecture, which is equivalent to the natural sleep architecture (i.e., when no medicaments are administered). There was no impairment of slow wave sleep, and only a small effect on REM sleep was observed. These results are demonstrated by the chart in FIG. 14.

Maintaining normal sleep architecture is a very important component of getting a good night rest. Some conventional insomnia medications, such as classic benzodiazepines, may have the ability to induce and maintain sleep, but they do so by considerably altering the normal sleep architecture, which results in unrefreshing sleep and other side effects.

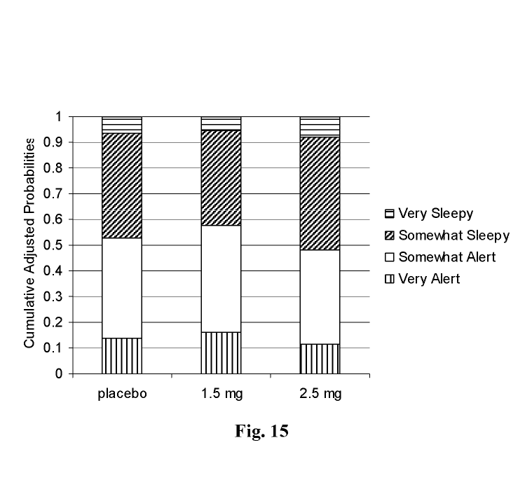

The results of the study conducted in accordance with Example 3 showed that the compound of formula (II) produces no patient-reported residual sedation effects at either the 1.5 mg dose or the 2.5 mg dose compared with the placebo. This is demonstrated by the chart in FIG. 15.

The objective residual effects of the administration of the compound of formula (II) were also evaluated. The scores on the Digit Symbol Substitution Test (DSST) taken by the subjects 9 hours after administering the dose were only slightly lower than those obtained from the subjects who were administered the placebo.

The results also showed that the compound of formula (II) was safe and well-tolerated at 1.5 mg and 2.5 mg doses. No serious side effects, and only a low incidence of adverse events, were reported. These results are summarized in Table 8.

TABLE 8
Safety Results

| | Placebo | 1.5 mg | 2.5 mg |
|---|---|---|---|
| Adverse Event | (n* = 70) | (n* = 71) | (n* = 71) |
| Number of Patients with Any | 12 (17.1%) | 13 (18.3%) | 18 (25.4%) |
| Event^{†} | | | |
| Number of Patients with | 3 (4.3%) | 3 (4.2%) | 6 (8.5%) |
| Headache | | | |
| Number of Patients with | 0 (0%) | 2 (2.8%) | 4 (5.6%) |
| Somnolence | | | |

*“n” refers to the total number of patients enrolled in the study
^{†}reported at any time in the study irrespective of whether these events were considered related to the medication

While the invention has been described in conjunction with the detailed description thereof and the accompanying figures, the foregoing description is intended to illustrate and not limit the scope of the invention, which is defined by the scope of the appended claims. Other aspects, advantages, and modifications are within the scope of the following claims.

1. A method for treating maintenance insomnia in a human in need thereof comprising administering to the human from about 0.5 mg to about 5 mg of a compound of formula (II) or a pharmaceutically acceptable salt thereof to treat the maintenance insomnia

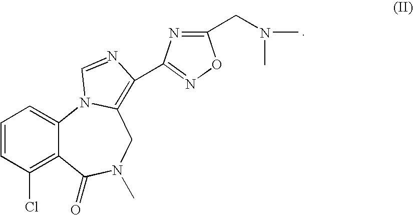

2. (canceled)

3. The method according to claim 1, wherein the amount of the compound of formula (II) is from about 1 mg to about 3 mg.

4. The method according to claim 1, wherein the amount of the compound of formula (II) is from about 1.5 mg to about 2.5 mg.

5. The method according to claim 1, wherein the administration of the compound of formula (II) or the pharmaceutically acceptable salt thereof is such as to also treat sleep onset insomnia,

6. The method according to claim 1, wherein the administration of the compound of formula (II) or the pharmaceutically acceptable salt thereof is such as to reduce latency to sleep onset, latency to persistent sleep and/or total wake time per hour during first eight hours after the administration.

7. The method according to claim 1, wherein the administration of the compound of formula (II) or the pharmaceutically acceptable salt thereof is such as to achieve a maximal potentiation of a response mediated by α₁-subunit containing GABA_{A} receptors from about 40% to about 90%.

8. A method for decreasing wake after sleep onset in a human in need thereof comprising administering to the human from about 0.5 mg to about 5 mg of a compound of formula (II) or a pharmaceutically acceptable salt thereof to decrease the wake after sleep onset

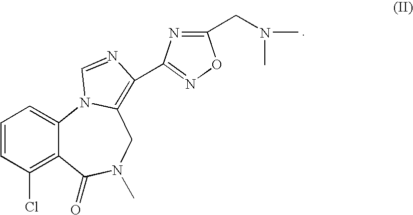

9. (canceled)

10. The method according to claim 8, wherein the amount of the compound of formula (II) is from about 1 mg to about 3 mg.

11. The method according to claim 8, wherein the amount of the compound of formula (II) is from about 1.5 mg to about 2.5 mg.

12. The method according to claim 8, wherein the administration of the compound of formula (II) or the pharmaceutically acceptable salt thereof is such as to also treat sleep onset insomnia;

13. The method according to claim 8, wherein the administration of the compound of formula (II) or the pharmaceutically acceptable salt thereof is such as to reduce latency to sleep onset, latency to persistent sleep and/or total wake time per hour during first eight hours after the administration.

14. The method according to claim 8, wherein the administration of the compound of formula (II) or the pharmaceutically acceptable salt thereof is such as to achieve a maximal potentiation of a response mediated by α₁-subunit containing GABA_{A} receptors from about 40% to about 90%.

15. A method for treating terminal insomnia in a human in need thereof comprising administering to the human from about 0.5 mg to about 5 mg of a compound of formula (II) or a pharmaceutically acceptable salt thereof to treat the terminal insomnia

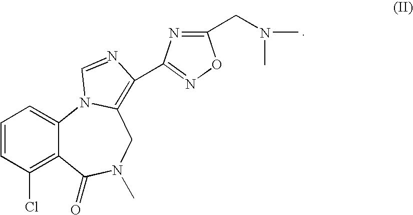

16. (canceled)

17. The method according to claim 15, wherein the amount of the compound of formula (II) is from about 1 mg to about 3 mg.

18. The method according to claim 15, wherein the amount of the compound of formula (II) is from about 1.5 mg to about 2.5 mg.

19. The method according to claim 15, wherein the administration of the compound of formula (II) or the pharmaceutically acceptable salt thereof is such as to also treat sleep onset and/or maintenance insomnia.

20. The method according to claim 15, wherein the administration of the compound of formula (II) or the pharmaceutically acceptable salt thereof is such as to reduce latency to sleep onset, latency to persistent sleep, wake after sleep onset and/or total wake time per hour during first eight hours after the administration.

21. The method according to claim 15, wherein the administration of the compound of formula (II) or the pharmaceutically acceptable salt thereof is such as to achieve a maximal potentiation of a response mediated by α₁-subunit containing GABA_{A} receptors from about 40% to about 90%.

22-28. (canceled)

29. A method for treating maintenance and/or terminal insomnia in a human in need thereof comprising administering to the human from about 0.5 mg to about 5 mg of a compound of formula (II) or a pharmaceutically acceptable salt thereof to achieve an AUC from about 17.5 ng•h/mL to about 600 ng•h/mL and a Cₘₐₓ from about 2.5 ng/mL to about 125 ng/mL

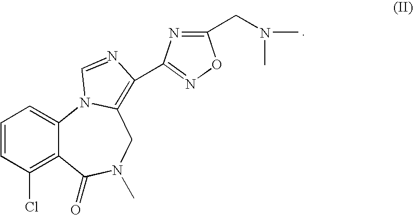

30. The method according to claim 29, wherein the AUC is from about 50 ng•h/mL to about 360 ng•h/mL.

31. The method according to claim 29, wherein the AUC is from about 75 ng•h/mL to about 240 ng•h/mL.

32. The method according to claim 29, wherein the Cₘₐₓ is from about 10.5 ng/mL to about 75 ng/mL.

33. The method according to claim 29, wherein the Cₘₐₓ is from about 15 ng/mL to about 45 ng/mL.

34. The method according to claim 29, wherein the administration of the compound of formula (II) or the pharmaceutically acceptable salt thereof is such as to also treat sleep onset insomnia,

35. The method according to claim 29, wherein the administration of the compound of formula (II) or the pharmaceutically acceptable salt thereof is such as to reduce latency to sleep onset, latency to persistent sleep, wake after sleep onset and/or total wake time per hour during first eight hours after the administration.

36. The method according to claim 29, wherein the administration of the compound of formula (II) or the pharmaceutically acceptable salt thereof is such as to achieve a maximal potentiation of a response mediated by α₁-subunit containing GABA_{A} receptors from about 40% to about 90%.

37-39. (canceled)
